(19) United States
(12) Patent Application Publication
(10) Pub. No.: US 2011/0236992 A1
(43) Pub. Date: Sep. 29, 2011

(54) METHOD OF DETECTING A SPECIFIC NUCLEOPHILE AND SURFACE ACOUSTIC WAVE SENSOR FOR DETECTING THE SPECIFIC NUCLEOPHILE

(75) Inventors: Soo Suk LEE, Suwon-si (KR); Yeol Ho LEE, Seoul (KR); Min Su HAN, Seoul (KR)

(73) Assignees: SAMSUNG ELECTRONICS CO., LTD., Suwon-si (KR); CHUNG-ANG UNIVERSITY INDUSTRY-ACADEMY COOPERATION FOUNDATION, Seoul (KR)

(21) Appl. No.: 13/035,350

(22) Filed: Feb. 25, 2011

(30) Foreign Application Priority Data
Mar. 25, 2010 (KR) 10-2010-0026887
Jul. 29, 2010 (KR) 10-2010-0073112

(51) Int. Cl. G01N 33/53; G01N 21/75; B82Y 15/00

(52) U.S. Cl. 436/501; 422/69; 422/400; 977/773; 977/810; 977/896; 977/902

(57) ABSTRACT

Provided herein is a method of detecting the presence of a specific nucleophile, which uses a material Y degraded by nucleophilic substitution of reacting with a specific nucleophile X, and a material Z selectively binding to a material Y′ produced by the degradation. According to the method, the nucleophile X can be easily analyzed according to the bonding between the materials Y′ and Z.

### CROSS-REFERENCE TO RELATED APPLICATION

This application claims priority to Korean Patent Application No. 10-2010-0026887, filed on Mar. 25, 2010, and Korean Patent Application No. 10-2010-0073112, filed on Jul. 29, 2010, and all the benefits accruing therefrom under 35 U.S.C. §119, the contents of which in their entirety are herein incorporated by reference.

### BACKGROUND

1. Field

The disclosure relates to a method of detecting a specific nucleophile, and a surface acoustic wave sensor for detecting the specific nucleophile.

2. Description of the Related Art

A surface acoustic wave (“SAW”) is a mechanical wave (in contrast to an electromagnetic wave) that is generated from movement of particles due to external thermal, mechanical, and/or electrical forces. In a SAW, a portion of a vibrational energy wave is concentrated on the surface of a medium. SAW sensors using the SAW include devices that sense a target material or its properties.

Generally, the SAW sensor is disposed on a substrate formed of a piezoelectric material, and includes a receptor that selectively binds to a target material on a surface thereof. Thus, when a sample containing the target material contacts the SAW sensor, there occurs a change in wavelength due to a physical, chemical and/or electrical reaction between the target material and the receptor. This change can be used to determine and/or monitor a content of the target material.

A nucleophile can be detected by selected ion monitoring (“SIM”) using high resolution gas chromatography-mass spectrometry (“GC/MS”). However, there remains a need for an improved method of selectively detecting a target material including a nucleophile.

### SUMMARY

Disclosed herein is a method of selectively detecting a specific nucleophile X.

In an aspect, the method of selectively detecting the presence or absence of a specific nucleophile X in a sample includes: contacting the sample with a receptor including a material Y which is selectively reactive with the specific nucleophile X, to form a material Y′; contacting the material Y′ with a material Z which selectively binds to the material Y′, to bond the material Z to the material Y′; and observing whether the bonding between the material Y′ and the material Z occurs or not to selectively detect the presence or absence of specific nucleophile X.

In the method, when the sample is contact with the receptor including the material Y degraded by reaction with the specific nucleophile X, if the specific nucleophile X is present in the sample, the material Y may be converted into a material Y′. The presence of the specific nucleophile X can be confirmed by observing the conversion of the material Y to Y′. The conversion of the material Y may be confirmed by reaction of y′ with the material Z, and observing whether bonding between the material Y′ and the material Z occurs or not.

The bonding between the material Y′ and the material Z may be determined by observing a change caused by the bonding of the material Y′ and the material Z. Examples of the change may include a change in color, mass, mechanical signal, or polarization characteristic. In an example, the change in mass caused by the bonding between the material Y′ and the material Z may be observed by detecting a frequency shift of a SAW sensor. In an embodiment, the bonding between the material Z and the material Y′ may be determined by observing the change in mass in accordance with the addition of the material Z to the receptor, and thus the material Z can have a detectable mass. The nucleophile X may be regenerated by hydrolysis.

An exemplary embodiment also provides a SAW sensor for selectively detecting a specific nucleophile. In an aspect, the sensor includes: a piezoelectric substrate; a first and a second inter-digitated transducer (IDT) disposed on the substrate; and a reaction layer disposed on the piezoelectric substrate and covering the first and the second IDT, and further having a receptor selectively reactive with a specific nucleophile X to be detected. In an aspect, the receptor is selectively reactive with the nucleophile to form a thiol, and wherein a metal nanoparticle selectively binds to the thiol.

The receptor may include a compound having a thiol group provided by nucleophilic substitution with a specific nucleophile, and a metal nanoparticle may bind to the compound having the thiol group. In an example, the receptor may include a thioester, the specific nucleophile may be CN⁻, and the metal nanoparticle may be an adenosine-5′-triphosphate stabilized gold (Au) nanoparticle.

The substrate may be subjected to a surface treatment to effectively attach the receptor. A surface-treating agent may be suitably selected depending on the receptor. For example, when the receptor includes an organic polymer, the receptor may be a silane that forms a self-assembled monolayer such as 3-mercaptopropyltrimethoxysilane (“3-MTPS”).

Also disclosed is a sensor for detecting a cyanide ion (CN⁻), the sensor including: a reaction unit having a receptor reactive with a cyanide ion (CN⁻), wherein the receptor includes a thioester convertible into an alkanethiol by nucleophilic substitution with the cyanide ion (CN⁻), and the alkanethiol is reactive with an adenosine-5′-triphosphate stabilized gold (Au) nanoparticle to change a color of the gold nanoparticle or a mass of the receptor.

When the adenosine-5′-triphosphate stabilized gold (Au) nanoparticle binds to the alkanethiol, the color of the gold nanoparticle may turn from red to blue, and such color change may be detected by measuring absorption of blue light or a ratio of absorption of red light to absorption of blue light.

### BRIEF DESCRIPTION OF THE DRAWINGS

The above and other aspects of the inventive concept will become more readily apparent by describing in further detail example embodiments thereof which reference to the accompanying drawings, in which:

FIG. 1 is a diagram illustrating an embodiment of a process of selectively detecting a specific nucleophile;

FIG. 2 is a diagram illustrating an embodiment of a reaction of a cyanide ion with a thioester;

FIG. 3 is diagram of an embodiment of a SAW sensor;

FIG. 4 is a graph of frequency shift (Hertz, Hz) versus a concentration of cyanide ions (picomolar, pM) according to Experimental Example 1;

FIG. 5 is a graph of frequency shift (Δf, Hertz) versus log cyanide concentration (log [CN], picomolar, pM) showing the log of the concentration of cyanide ions of FIG. 4;

FIG. 6 is a graph of frequency shift (Δf, Hertz) versus a type of anion according to Experimental Example 2; and

FIG. 7 is a graph of a ratio of absorption at 680 nm to an absorption at 520 nm (an A₆₈₀/A₅₂₀ level) versus a type of anion according to Experimental Example 3.

### DETAILED DESCRIPTION

The invention now will be described more fully hereinafter with reference to the accompanying drawings, in which a non-limiting embodiment is shown. This invention may, however, may be embodied in many different forms, and should not be construed as limited to the embodiments set forth herein. Rather, these embodiments are provided so that this disclosure will be thorough and complete, and will fully convey the scope of the invention to those of ordinary skill in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present invention.

The terminology used herein is for the purpose of describing particular embodiments only and is not intended to be limiting. As used herein, the singular forms “a,” “an,” and “the” are intended to include the plural forms as well, unless the context clearly indicates otherwise. It will be further understood that the terms “comprises” and/or “comprising,” or “includes” and/or “including” when used in this specification, specify the presence of stated regions, integers, steps, operations, elements, and/or components, but do not preclude the presence or addition of one or more other regions, integers, steps, operations, elements, components, and/or groups thereof.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

### Method of Detecting a Specific Nucleophile

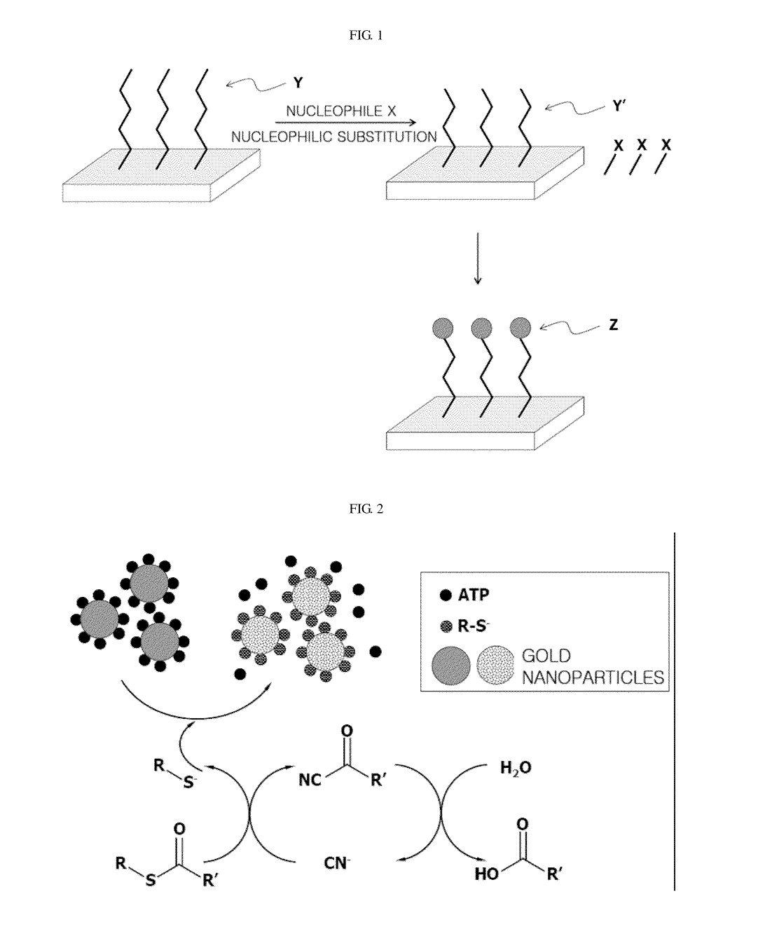

FIG. 1 is a schematic diagram illustrating an embodiment of a method of selectively detecting the presence or absence of a specific nucleophile in a sample.

Referring to FIG. 1, the detecting method according to an embodiment includes: contacting a sample with a material Y, which is selectively reactive with a specific nucleophile X to form a material Y′; contacting the material Y′ with a material Z, which selectively binds to the material Y′ to form a bond between the material Z and the material Y′; and observing whether the bond between the material Y′ and the material Z occurs or not, to selectively detect the presence or absence of the specific nucleophile in the sample.

In other words, when the material Y′ binds to the material Z, it can be determined that the specific nucleophile X is present in the sample by observing such binding. However, when the material Y′ and the material Z do not bind to each other, it can be determined that the specific nucleophile X is substantially or entirely absent from the sample.

The reaction of the specific nucleophile X with the material Y may be performed simultaneously with the reaction of the material Z with the material Y′. Accordingly, t specific nucleophile X, the material Y, and the material Z may be simultaneously present.

In the detecting method according to an embodiment, when a material, e.g., material Y reactive with a specific nucleophile X contacts a sample to be analyzed, the material Y will selectively react with the specific nucleophile X if the specific nucleophile is present in the sample. Thus, the presence of the specific nucleophile X can be determined by observing whether or not the material Y is present or has been converted to the material Y′. The conversion of the material Y may be determined by observing whether a material Y′, which is a reaction product of the material Y, reacts or does not react with the material Z, wherein the material Z selectively binds to the material Y′.

While not wanting to be bound by theory, it is understood that the specific nucleophile has an electron-rich atom and may be negatively charged. Thus, in a formal sense, the nucleophile can form a chemical bond to a reaction partner by donating a pair of electrons to a positively-charged or electron-deficient atom. Examples of the specific nucleophile include an anion, such as a halide, e.g., I⁻, Cl⁻, or Br⁻, a hydroxyl ion (OH⁻), or a cyanide ion (CN⁻), or ammonia (NH₃).

Among the foregoing specific nucleophiles, the cyanide ion (CN⁻) is very toxic, and can be adsorbed to a heme group of a cytochrome which can impair proper functioning of a living body when it accumulates in the living body through aspiration and/or transcutaneously, for example, resulting in inactivation of the cytochrome. Due to such strong toxicity, when the cyanide ion binds to a red blood cell heme, hypoxia can result, which leads to death of the animal or human.

The cyanide ion (CN⁻) exhibits toxicity even at a very low concentration, e.g., a concentration of about 0.01 milligrams per liter (mg/L), which is less than lead (e.g., about 0.05 mg/L) or arsenic (e.g., about 0.05 mg/L), which are also highly toxic, according to a standard for water quality management. Because cyanide ions (CN⁻) are produced via various routes, for example, from an industrial process such as mining and/or plating, and from various industrial wastes, it would be desirable to simplify monitoring of cyanide.

The bonding of the material Y′ to the material Z may be observed by a change caused by the bonding between the materials Y′ and Z. For example, the change may include a change in a color or a mass, as well as change in a mechanical signal or a polarization characteristic.

In an embodiment, when the material Y is a thioester and it is converted into an alkanethiol (material Y′) by reaction with a cyanide ion (specific nucleophile X), the alkanethiol (material Y′) may be precipitated in H₂O by reaction with a stabilized gold nanoparticle (e.g., adenosine-5′-triphosphate (“ATP”) stabilized gold nanoparticle) (material Z), and the stabilized gold nanoparticle (material Z) may turn from red to blue upon addition of the material Y′. Accordingly, by observing the color change, the bonding between the materials Y′ and Z can be detected. In an embodiment, the alkane thiol may have from 1 to 16 carbon atoms.

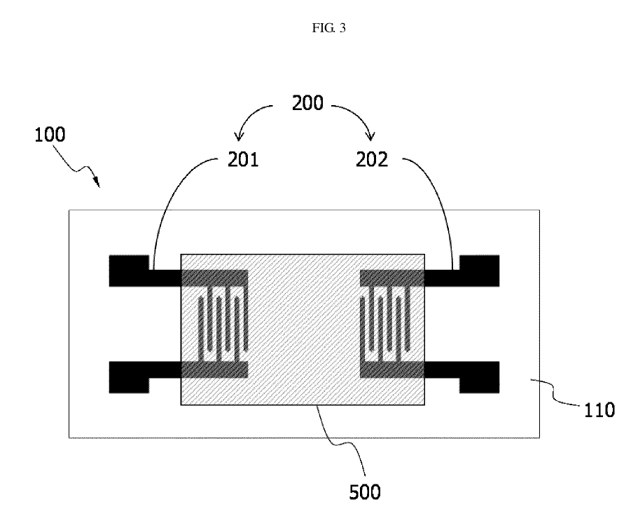

The foregoing process is shown schematically in FIG. 2. Referring to FIG. 2, and as shown in Reaction Scheme 1, when the cyanide ion reacts with the thioester, the thioester is converted into alkanethiol through nucleophilic substitution. After the reaction, the cyanide ion may be regenerated by hydrolysis.

### Reaction Scheme 1

R—SC(O)R′+CN⁻→CN—C(O)R′+(R—S—) [Nucleophilic Substitution]

The produced alkanethiol can bond with the stabilized gold nanoparticle, thereby obtaining an alkanethiol-bound to the gold nanoparticle, and the color of the gold nanoparticle may turn from red to blue. The stabilizer (e.g. ATP bound to the gold nanoparticle) may be completely or partially displaced by the alkanethiol, and in an embodiment the stabilizer on the gold nanoparticle is completely displaced. Thus, the material Z may be an ATP stabilized gold (Au) nanoparticle.

In another example, when the alkanethiol (material Y′) contacts (e.g., reacts with) the stabilized gold nanoparticle (material Z), the mass (e.g., gram molecular weight) of the gold nanoparticle (material Z) occurs. Thus, the bonding between the material Y′ and the material Z may be detected by observing the mass change. For example, the mass change due to the bonding between the material Y′ and the material Z may be observed by detecting a frequency shift of a SAW sensor. Herein, the material Z may have a mass detectable by the SAW sensor.

According to an embodiment of the detecting method, the following conditions are present: (i) the material Y is selectively reactive with the specific nucleophile X, and (ii) the material Z selectively binds to the material Y′. These conditions may be met by appropriate selection of materials, and/or control of the reaction conditions.

The disclosed method may be performed at about 0 to about 100° C., about 5 to about 70° C., or about 10 to about 40° C., or at room temperature (e.g., about 20° C.), and at a neutral pH (e.g. 7), or at a pH of about 6 to about 8, or about 6.5 to about 7.5, to prevent an additional reaction or continuous reaction. To this end, the method may be performed in a buffer.

In addition to the material Y′, the contacting of the nucleophile X and the material Y provides a residue comprising the nucleophile X. The residue may be reduced to provide the specific nucleophile X by hydrolysis. Thus, although the specific nucleophile X may be contained in the sample in a low concentration, the specific nucleophile X may further react with the material Y after the reduction, thereby further improving detection sensitivity.

### SAW Sensor for Detecting a Specific Nucleophile

According to an aspect, a SAW sensor for selectively detecting a specific nucleophile is provided. The SAW sensor is capable of analyzing properties of a target material such as mass, pressure, density, and viscosity. Further, the SAW sensor can be easily integrated into a device, can have a compact size, and can measure a small quantity of sample in real time.

For example, the SAW sensor may convert the subtle change in mass caused by interaction between molecules on a surface of the sensor into a detectable frequency shift or phase shift. In the SAW sensor, an electrical signal generates a mechanical wave through an inter-digitated transducer (“IDT”) on the surface of the SAW sensor, and the wave is changed by physical, chemical, or mechanical reactions of the surface of the SAW sensor with a target nucleophile. The SAW sensor measures the change in a wave (e.g., frequency or amplitude) to provide qualitative and/or quantitative analysis of the target nucleophile. To generate the wave of the SAW sensor, a resonator and/or an oscillator may use an oscillation method of applying an output signal of the SAW sensor to an input signal of the SAW sensor, or a method of generating a specific frequency outside the SAW sensor, applying the frequency to the input IDT of the SAW sensor, and plotting the output signal.

The SAW sensor may be used to detect any material having a mass. For example, when a heavy metal ion, a toxic anion, or a material such as a toxin binds to the surface of the sensor, a surface mass of the sensor is changed due to the material binding to the surface of the sensor, resulting in a change in a signal of the sensor. Thus, the sensor can determine the presence and content of the target material.

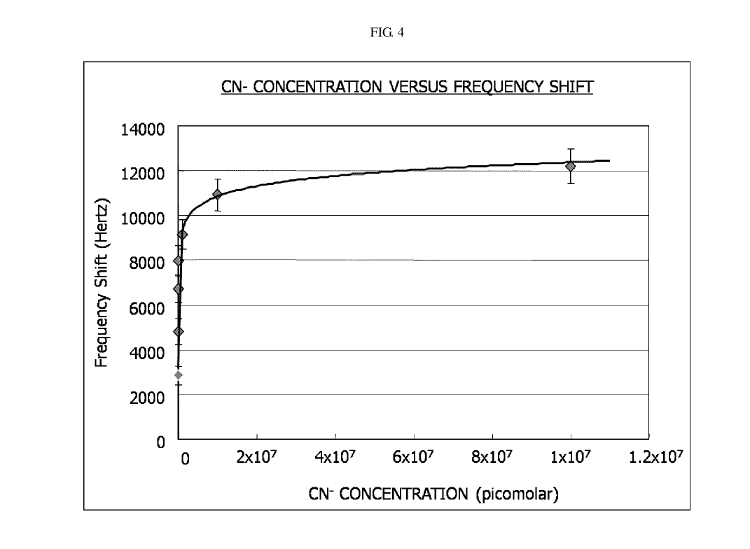

FIG. 3 is a schematic diagram of an embodiment of a SAW sensor. Referring to FIG. 3, the SAW sensor 100 includes a piezoelectric substrate 110, first and second interdigitated transducers (“IDTs”) 201 and 202, respectively, disposed on the substrate; and a reaction layer 500 disposed on the piezoelectric substrate 110 to fully or partially cover the first and second IDTs 201 and 202. The reaction layer 500 may and bind to the target material to be detected or comprise a receptor that binds to the target material to be detected.

The receptor may include a compound in which a thiol group is provided by nucleophilic substitution of a thioester group with a specific nucleophile. A metal nanoparticle may bind to the compound having the thiol group.

The substrate 100 comprises a piezoelectric material whose electrical characteristics change when a mechanical signal is applied (e.g., via the piezoelectric effect), or generate a mechanical signal when an electrical signal is applied (e.g., via the reverse piezoelectric effect). For example, the piezoelectric material may include lithium niobate (LiNbO₃), lithium tantalate (LiTaO₃), lithium tetraborate (Li₂B₄O₇), barium titanate (BaTiO₃), PbZrO₃, PbTiO₃, PZT, ZnO, GaAs, quartz, niobate. A combination comprising at least one of the foregoing may be used.

The surface of the substrate may be treated with a material capable of binding to a receptor, a material to be bound to the receptor, or a material capable of forming a self-assembled monolayer (“SAM”), such as 3-mercaptopropyltrimethoxysilane (“3-MTPS”).

The pair of interdigitated transducers (“IDT”) 200 may be an interface between an electrical circuit and an acoustic delay line. The pair of IDTs 200 includes first and second IDTs 201 and 202, respectively. Of the pair of IDTs 200, the first IDT 201 may generate a surface acoustic wave in response to an applied signal, and may be referred to as an “input IDT” or as a “transducer.” The surface acoustic wave propagates to the second IDT 202 by expansion and compression with an appropriate frequency along the surface of the substrate, and converted into an electrical signal due to the reverse piezoelectric effect. The second IDT 202 may be referred to as an “output IDT” or as a “receiver.”

The pair of IDTs 200 may comprise aluminum (Al) or an Al alloy. The Al alloy may include Al as a main component and at least one of Ti, Si, Cr, W, Fe, Ni, Co, Pb, Nb, Ta, Zn, or V.

The receptor may comprise a material reactive with the specific nucleophile by the nucleophilic substitution, for example, a compound having a thiol group that results from the nucleophilic substitution. The metal nanoparticle binds to the thiol group, and the SAW sensor detects the change in mass caused by the binding of the metal nanoparticle to the receptor.

In an embodiment, the SAW sensor may selectively detect a cyanide ion as the specific nucleophile.

Commercially, the cyanide ion (CN⁻) is generally detected by a selected ion monitoring (“SIM”) method using high resolution gas chromatography-mass spectrometry (“GC/MS”). However, while GC/MS is very effective, a GC/MS includes an expensive apparatus and cannot readily measure as sample in real time. Also a GC/MS is often operated by a highly-skilled technician. For reasons such as these, there remains a need for a detecting method that can be easily used by an untrained technician. According to an embodiment, the SAW sensor can determine the presence of the cyanide ion (CN⁻), and can also perform quantitative analysis.

The SAW sensor for detecting the cyanide ion may comprise a receptor comprising the thioester and a stabilized (e.g., ATP stabilized) gold (Au) nanoparticle as the metal nanoparticle.

The thioester is converted into an alkanethiol by nucleophilic substitution with the cyanide ion (CN⁻). The alkanethiol comprises a thiol group, may be attached to the surface of the sensor by a covalent bond, is reactive with the CN⁻ ion, and binds to the stabilized metal nanoparticle. Thereby, a dramatic change in mass may be induced due to sorption (e.g., adsorption) of the gold particle to the surface of the SAW sensor. The mass change leads to a change in a signal of the SAW sensor, and thus may provide quantitative measurement of the concentration of the specific nucleophile.

Herein, a sample for analysis containing the cyanide ion (CN⁻) and the metal nanoparticle may be sequentially or simultaneously added.

According to an operating principle of the SAW sensor, a mechanical wave is generated when an electrical signal goes through the input IDT 201. The wave is changed by a physical, chemical, or electrical reaction occurring when the receptor included in the reaction layer 500 on the surface of the SAW sensor binds to a target material. In other words, a center frequency, phase, or size of an output signal of the SAW sensor may be changed. Therefore, observation of the signal change may provide detection of the target material binding to the SAW sensor and further provide qualitative and quantitative analyses of the target material comprising the specific nucleophile.

When a weight of the reaction layer 500 is changed due to introduction of a specific nucleophile to the reaction layer 500 and binding of the metal nanoparticle by nucleophilic substitution, a shear rate of the SAW subjected to induced vibration by the IDT 201 is changed, and an oscillation frequency shift is measured by the IDT 202 receiving and using the change in shear rate, thereby precisely detecting the SAW.

### Sensor for Detecting Cyanide Ion (CN⁻)

According to another embodiment, provided is a sensor for detecting cyanide ion (CN⁻). The sensor includes a reaction unit to which a receptor reactive with a target material, i.e., the cyanide ion (CN⁻), is bound. The receptor may be a thioester, which is converted into an alkanethiol by nucleophilic substitution with the cyanide ion (CN⁻). The sensor can detect the cyanide ion (CN⁻) according to a change in color or mass that occurs when the alkanethiol reacts with a stabilized (e.g., ATP stabilized) gold nanoparticle.

In this embodiment, when the gold nanoparticle binds to the alkanethiol, the sensor for detecting the cyanide ion (CN⁻) detects a change in color or mass. Thus conversion of thioester into alkanethiol by the cyanide ion (CN⁻) may lead to binding of the gold nanoparticle. Therefore, the sensor can detect the presence of the cyanide ion as well as perform quantitative analysis thereof.

The change in color can be observed by eye, or may be detected by absorption. For example, when the alkanethiol binds to the metal nanoparticle, the color may turn from red to blue. Such a change in color can be detected by quantifying the absorption of blue light. Also, the change in color may be detected by an absorption ratio of red light to blue light.

The change in mass may be detected using various known mass sensors, which may include a surface plasmon resonance (“SPR”) sensor, a quartz crystal microbalance (“QCM”) sensor, a cantilever sensor, or a bulk acoustic wave (“BAW”) sensor, as well as the SAW sensor.

The SPR sensor uses a standard measurement principle for measuring a degree of absorption of a sample on a surface of a bulk metal (e.g., gold or silver) or metal nanoparticle. SPR refers to a state of surface plasmons excited by light incident onto a planar surface.

The QCM sensor measures a concentration of a target material by immobilizing a receptor onto a quartz crystal coated with a coupling agent, reacting with a specific material to be measured, and measuring a variation in frequency before and after the reaction.

The cantilever sensor uses bending of a cantilever caused by a change in a resonance frequency and stress when a molecule is adsorbed onto the surface of the cantilever.

The SAW sensor and the BAW sensor sense the presence or properties of a target material using an acoustic wave propagated as an elastic wave in a solid. The acoustic wave is a mechanical wave (in contrast with an electromagnetic wave) generated by movement of particles by external thermal, mechanical and/or electrical forces, and a large portion of vibrational energy is concentrated on the surface of a medium. BAW is propagated through the bulk of an elastic substrate, and SAW is propagated along the surface of the substrate.

Hereinafter, an embodiment will be disclosed in further detail with reference to Experimental Examples.

### Experimental Example 1

A SAM is formed by treating the surface of a SAW sensor having a SiO₂ layer with 3-mercaptopropyltrimethoxysilane (“3-MTPS”), and contacted with acetic anhydride in the presence of KHCO₃ in a dry medium for acetylation.

The surface-treated SAW sensor comprising a thioester on its surface is contacted with cyanide ion, thereby providing a thiol group. The thiol group on the surface of the SAW sensor is reacted with an adenosine-5′-triphosphate (“ATP”) stabilized gold nanoparticle, thereby inducing a dramatic change in mass caused by adsorption of the gold nanoparticle to the surface of the SAW sensor, resulting in a change in signal on the surface of the sensor.

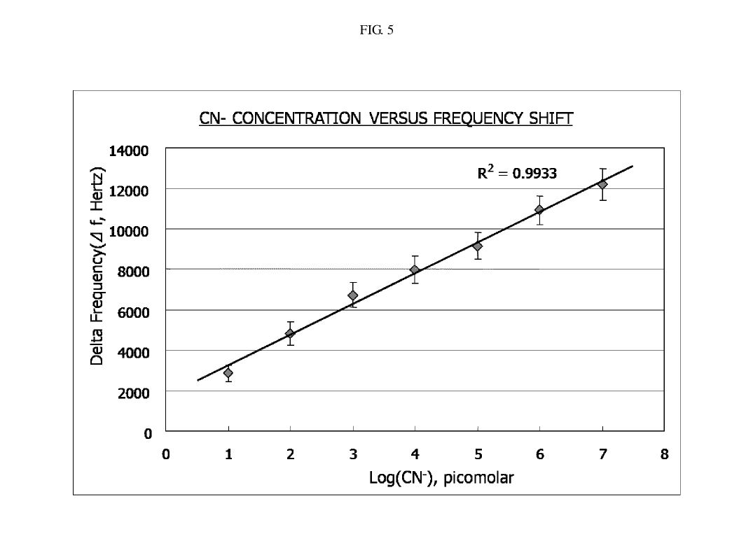

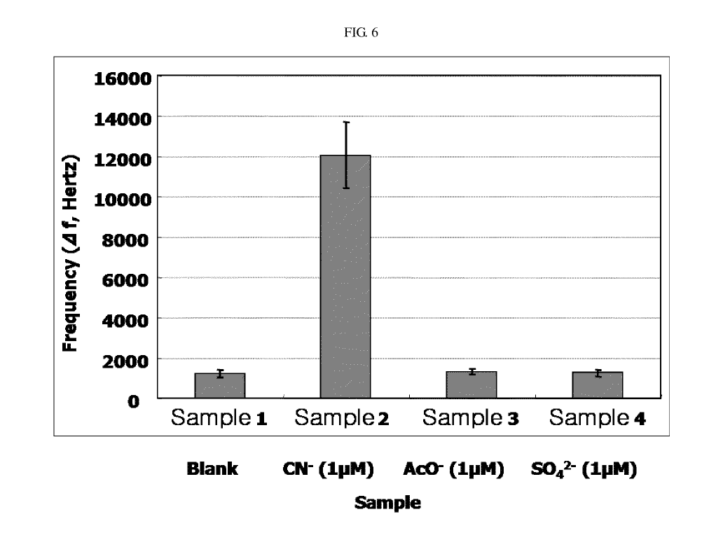

A concentration of the cyanide ion is measured by adding various concentrations of the cyanide ion (CN⁻) and measuring a frequency shift of the SAW sensor, and the results are shown in FIGS. 4 and 5.

Referring to FIG. 4, as the content of CN⁻ is increased, the frequency shift became greater, and saturation is observed at a certain concentration. Referring to FIG. 5, a graph of frequency versus log concentration provides straight line. A measurement limit of CN⁻ of the sensor is about 1.0 picomolar (pM).

### Experimental Example 2

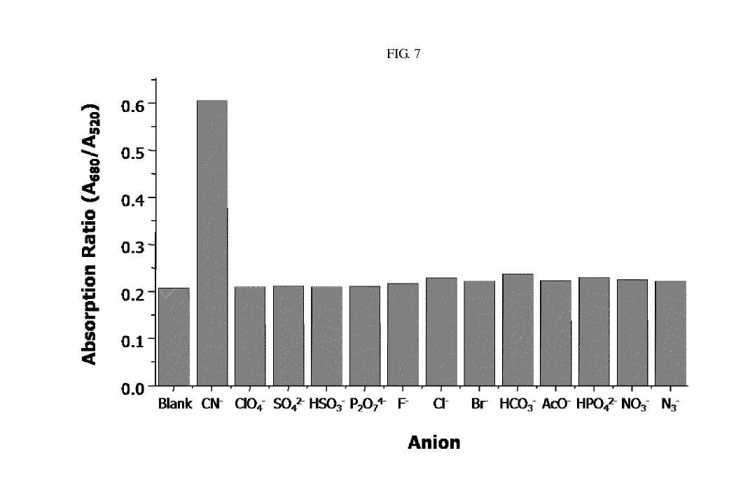

A 1 micromolar (μM) quantity of each of phosphate buffered saline (“PBS”) buffer (sample 1), CN⁻ (sample 2), AcO⁻ (sample 3), and SO²⁻ (sample 4) is added to the SAW sensor used in Experimental Example 1, and frequency shifts are measured by the SAW sensor. The results are shown in FIG. 6.

Referring to FIG. 6, when CN⁻ is used, a frequency shift occurred at 12,000 Hz, and in the cases of samples 3 and 4, frequencies are the same as that of sample 1.

### Experimental Example 3

Samples are prepared by adding 20 millimolar (mM) of selected anions, specifically CN⁻, ClO₄⁻, SO₄²⁻, HSO₃⁻, P₂O₇⁴⁻, F⁻, Cl⁻, Br⁻, HCO₃⁻, AcO⁻, HPO₄²⁻, NO₃⁻, or N³⁻ to solutions each containing 3 nanomolar (nM) of a gold nanoparticle, 50 μM ATP, 1 mM PBS (pH 7.0), and 1 mM thioester, and an anion-free sample (blank). Here, examples of anions include CN⁻, F⁻, Cl⁻, Br⁻, ClO₄⁻, HSO₃⁻, SO₄²⁻, HCO₃⁻, AcO⁻, HPO₄²⁻, NO₃⁻, N^{3 -}, and P₂O₇⁴⁻.

As a result, at pH 7.0, only the CN-containing sample turns blue, while other samples are red, which is observed by eye.

To confirm the above results, light absorption of the samples was measured, and a ratio of absorption at 680 nanometers (nm) (e.g., absorbing red light) to absorption at 520 nm (e.g., absorbing blue light) is shown in FIG. 7.

As shown in FIG. 7, while a high ratio of the absorption at 680 nm to the absorption at 520 nm (i.e., A₆₈₀/A₅₂₀) was shown in the CN⁻-containing sample, almost the same levels of A₆₈₀/A₅₂₀ of about 0.2 were present in the other samples.

While exemplary embodiments have been disclosed herein, it should be understood that other variations are possible. Such variations are not to be regarded as a departure from the spirit and scope of exemplary embodiments of the present application, and all such modifications are intended to be included within the scope of the following claims.

What is claimed is:

1. A method of selectively detecting the presence or absence of a specific nucleophile in a sample, the method comprising:
contacting the sample with a receptor comprising a material Y which is selectively reactive with the specific nucleophile X, to form a material Y′;
contacting the material Y′ with a material Z which selectively binds to the material Y′ to bond the material the material Y′ to the material Z; and
observing whether the bonding between the material Y′ and the material Z occurs or not, to selectively detect the presence or the absence of the specific nucleophile X.

2. The method of claim 1, wherein the bonding between the material Y′ and the material Z is observed by observing a change in color caused by the bonding between the material Y′ and the material Z.

3. The method of claim 1, wherein the bonding between the material Y′ and the material Z is observed by a observing a change in mass caused by the bonding between the material Y′ and the material Z.

4. The method of claim 3, wherein the change in mass is observed by a change in frequency of a surface acoustic wave sensor.

5. The method of claim 1, wherein the contacting the sample with a receptor comprising a material Y and the contacting the material Y′ with a material Z are each independently performed at a pH of about 6 to about 8.

6. The method of claim 1, wherein the nucleophile X is cyanide ion, the material Y is thioester, the material Y′ is an alkanethiol, and the material Z is an adenosine-5′-triphosphate stabilized gold nanoparticle.

7. The method of claim 1, further comprising forming the specific nucleophile X by hydrolysis of a product of the contacting a material Y with a specific nucleophile X.

8. The method of claim 1, wherein the contacting a material Y comprises nucleophilic substitution of Y with specific nuclophile X.

9. A surface acoustic wave sensor for selectively detecting a specific nucleophile, the surface acoustic wave sensor comprising:
a piezoelectric substrate;
a first and a second inter-digitated transducer disposed on the substrate; and
a reaction layer disposed on the piezoelectric substrate and covering the first and the second inter-digitated transducers, and comprising a receptor selectively reactive with a specific nucleophile X to be detected,
wherein the receptor is selectively reactive with the nucleophile to form a thiol, and
wherein a metal nanoparticle selectively binds to the thiol.

10. The surface acoustic wave sensor of claim 9, wherein the nucleophile is a cyanide ion, the receptor is thioester, and the metal nanoparticle is an adenosine-5′-triphosphate stabilized gold nanoparticle.

11. The surface acoustic wave sensor of claim 9, wherein the substrate is a 3-mercaptopropyltrimethoxysilane surface treated substrate.

12. The surface acoustic wave sensor of claim 9, wherein the thiol is an alkanethiol.

13. A sensor for detecting a cyanide ion, the sensor comprising:
a reaction unit having a receptor reactive with a cyanide ion,
wherein the receptor comprises a thioester convertable into an alkanethiol by nucleophilic substitution with the cyanide ion, and the alkanethiol is reactive with an adenosine-5′-triphosphate stabilized gold nanoparticle to change a color of the gold nanoparticle or a mass of the receptor.

14. The sensor of claim 13, wherein the change in color is detected as an absorption of blue light, or a ratio of absorption of red light to absorption of blue light.
